(19) United States
(12) Patent Application Publication
(10) Pub. No.: US 2026/0000907 A1
(43) Pub. Date: Jan. 1, 2026

(54) METHODS, IMPLANTABLE PHOTOBIOMODULATION LEADS, AND SYSTEMS FOR IMPLANTATION DEPTH CONTROL

(71) Applicant: Boston Scientific Neuromodulation Corporation, Valencia, CA (US)

(72) Inventor: Joshua Dale Howard, Sacramento, CA (US)

(21) Appl. No.: 19/247,176

(22) Filed: Jun. 24, 2025

Related U.S. Application Data
(60) Provisional application No. 63/665,205, filed on Jun. 27, 2024.

(51) Int. Cl. A61N 5/06; A61B 34/20

(52) U.S. Cl. CPC A61N 5/0601; A61B 34/20; A61B 2034/2051; A61B 2034/2065; A61N 2005/0612

(57) ABSTRACT

A photobiomodulation lead can include a lead body; an enclosure coupled to the distal end portion of the lead body; a light emitter disposed in the enclosure and configured to emit light transmitted distally with a conical shape; and a radiopaque tip disposed distal to the light emitter, wherein the light emitter and radiopaque tip are spaced apart so that a portion of the light emitted with the conical shape passes distally beyond the radiopaque tip. An end of another photobiomodulation lead can be determined using a reference overlay by overlaying the reference overlay on an image of the photobiomodulation lead with first and second bounding lines aligned along opposing sides of the photobiomodulation lead in the image and a reference line positioned at a lead landmark visible in the image of the photobiomodulation lead. An end line indicates the position of the distal end of the photobiomodulation lead.

### CROSS-REFERENCE TO RELATED APPLICATIONS

This application claims the benefit under 35 U.S.C. § 119(e) of U.S. Provisional Patent Application Ser. No. 63/665,205, filed Jun. 27, 2024, which is incorporated herein by reference.

### FIELD

The present invention is directed to the area of implantable photobiomodulation (PBM) systems and methods of making and using the systems. The present invention is also directed to methods, implantable photobiomodulation leads, and systems for implantation depth control, as well as methods of making and using the leads and systems.

### BACKGROUND

Implantable photobiomodulation (PBM) systems can provide therapeutic benefits in a variety of diseases and disorders. PBM can provide treatment for symptoms, as well as full or partial relief from pain and other effects of the disease or disorder. For example, photobiomodulation can be applied to the brain either externally or using an implanted photobiomodulation lead to provide, for example, deep brain photobiomodulation, to treat a variety of diseases or disorders. A PBM system may include one or more light sources and, often, one or more optical fibers to carry the light to the desired modulation site. Photobiomodulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the brain structures, nerves, muscles, or other tissue to be stimulated.

### BRIEF SUMMARY

One aspect is a photobiomodulation lead that includes a lead body having a distal end portion and a proximal end portion; an enclosure coupled to the distal end portion of the lead body; a light emitter disposed in the enclosure and configured to emit light transmitted distally with a conical shape; and a radiopaque tip disposed distal to the light emitter, wherein the light emitter and radiopaque tip are spaced apart so that a portion of the light emitted with the conical shape passes distally beyond the radiopaque tip.

In at least some aspects, the light emitter includes a light source. In at least some aspects, the photobiomodulation lead further includes an optical waveguide extending along at least the distal end portion of the lead body, wherein the light emitter includes a distal end portion of the optical waveguide. In at least some aspects, the light emitter includes at least one of a prism, a plurality of mirrors, or a cylindrical lens for distributing the light in the conical shape.

Another aspect is a method of stimulating patient tissue of the patient. The method includes implanting any of the photobiomodulation leads described above into the patient tissue and emitting the light from the light emitter of the photobiomodulation lead to stimulate the patient tissue.

A further aspect is a photobiomodulation system that includes any of the photobiomodulation leads described above and a control module coupleable to the photobiomodulation lead. The control module includes a housing and an electronic subassembly disposed in the housing and configured to direct the emission of the light.

In at least some aspects, the photobiomodulation lead further includes an optical waveguide extending along at least the distal end portion of the lead body, wherein the light emitter includes a distal end portion of the optical waveguide, the photobiomodulation system including a light source configured for transmitting light into the optical waveguide when directed by the electronic subassembly of the control module. In at least some aspects, the light source is disposed in the control module. In at least some aspects, either a) the light source is disposed in the photobiomodulation lead or b) the photobiomodulation system further includes a lead extension coupleable to the photobiomodulation lead and the control module, wherein the light source is disposed in the lead extension.

Yet another aspect is a method for implanting a photobiomodulation lead. The method includes introducing the photobiomodulation lead into tissue of a patient; obtaining an image of the photobiomodulation lead in the tissue; providing a reference overlay including a first bounding line, a second bounding line parallel to the first bounding line, a reference line intersecting the first and second bounding lines, and an end line intersecting the first and second bounding lines; and determining a position of a distal end of the photobiomodulation lead by overlaying the reference overlay on the image with the first and second bounding lines aligned along opposing sides of the photobiomodulation lead in the image and the reference line positioned at a lead landmark visible in the image of the photobiomodulation lead, wherein the end line indicates the position of the distal end of the photobiomodulation lead.

In at least some aspects, the image is a fluoroscope image. In at least some aspects, the reference overlay is a physical transparency overlay. In at least some aspects, obtaining the image includes displaying the image on a display. In at least some aspects, providing a reference overlay includes generating the reference overlay on the display. In at least some aspects, the method further includes altering a scale of the image or the reference overlay so that the first and second bounding lines are aligned along opposing sides of the photobiomodulation lead.

Another aspect is a system for guiding implantation of a photobiomodulation lead into tissue of a patient. The system includes a display; a memory having instructions disposed thereon; and a processor coupled to the memory and display and configured to execute the instructions to perform actions, the actions including: obtaining an image of the photobiomodulation lead in the tissue, providing a reference overlay including a first bounding line, a second bounding line parallel to the first bounding line, a reference line intersecting the first and second bounding lines, and an end line intersecting the first and second bounding lines, and determining a position of a distal end of the photobiomodulation lead by overlaying the reference overlay on the image with the first and second bounding lines aligned along opposing sides of the photobiomodulation lead in the image and the reference line positioned at a lead landmark visible in the image of the photobiomodulation lead, wherein the end line indicates the position of the distal end of the photobiomodulation lead.

In at least some aspects, the system is an augmented reality system. In at least some aspects, the image is fluoroscope image. In at least some aspects, the actions further include altering a scale of the image or the reference overlay so that the first and second bounding lines are aligned along opposing sides of the photobiomodulation lead. In at least some aspects, the actions further include providing controls for a user to either a) move the reference overlay relative to the image or b) draw the reference overlay on the image.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of a photobiomodulation system that includes a lead electrically coupled to a control module;

FIG. 2A is a schematic side view of one embodiment of the control module of FIG. 1 configured and arranged to couple to an elongated device;

FIG. 2B is a schematic side view of one embodiment of a lead extension configured and arranged to couple the elongated device of FIG. 2A to the control module of FIG. 1;

FIG. 3A is a schematic side view of one embodiment of a distal end portion of a photobiomodulation lead;

FIG. 3B is a schematic view of an alternative embodiment of a light emitter that can be used with the photobiomodulation lead of FIG. 3A;

FIG. 3C is a schematic side view of another embodiment of a distal end portion of a photobiomodulation lead;

FIG. 4 is a schematic perspective exploded view of a further embodiment of a photobiomodulation lead;

FIG. 5A is a block diagram of one embodiment of a system for guiding implantation of a photobiomodulation lead into tissue of a patient;

FIG. 5B is a block diagram of another embodiment of a system for guiding implantation of a photobiomodulation lead into tissue of a patient;

FIG. 6A is a fluoroscopic image of distal ends of a photobiomodulation lead and an electrical stimulation lead;

FIG. 6B is an illustration of one embodiment of a reference overlay;

FIG. 6C illustrates the reference overlay of FIG. 6B disposed over a fluoroscopic image of distal ends of a photobiomodulation lead and an electrical stimulation lead; and

FIG. 7 is a schematic overview of one embodiment of components of a photobiomodulation system, including an electronic subassembly disposed within a control module.

### DETAILED DESCRIPTION

The present invention is directed to the area of implantable photobiomodulation (PBM) systems and methods of making and using the systems. The present invention is also directed to methods, implantable photobiomodulation leads, and systems for implantation depth control, as well as methods of making and using the leads and systems.

Examples of PBM and PBM/electrical stimulation systems can be found at, for example, U.S. Pat. Nos. 9,415,154; 10,335,607; and 10,814,140; U.S. Patent Applications Publications Nos. 2020/0155854; 2021/0008388; 2021/0008389; 2021/0016111; 2022/0072329; and 2024/0058619; U.S. patent application Ser. Nos. 18/232,621 and 18/397,740; and U.S. Provisional Patent Application Ser. Nos. 63/547,925 and 63/603,393, all of which are incorporated herein by reference in their entireties. Examples of electrical and PBM/electrical stimulation systems with leads that can be used or modified to include the elements described herein are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated herein by reference in their entireties.

Photobiomodulation can include photostimulation or optical stimulation. Photostimulation or optical stimulation can include generation of a neural signal (i.e., an electrical signal) or an action potential by the activation of neural tissue (e.g., neurons or other neural cells) in response to optical irradiation. PBM may include, but is not necessarily limited to, optical modulation, other modulation, or other effects resulting from biological response to particular wavelengths or wavelength ranges of light or from thermal effects generated using light or from any combination thereof. In at least some embodiments, the biological or cellular response includes modulation (for example, up-regulation or down-regulation) of one or more chemical or biological entities. In at least some embodiments, the photobiomodulation does not produce photostimulation or optical stimulation.

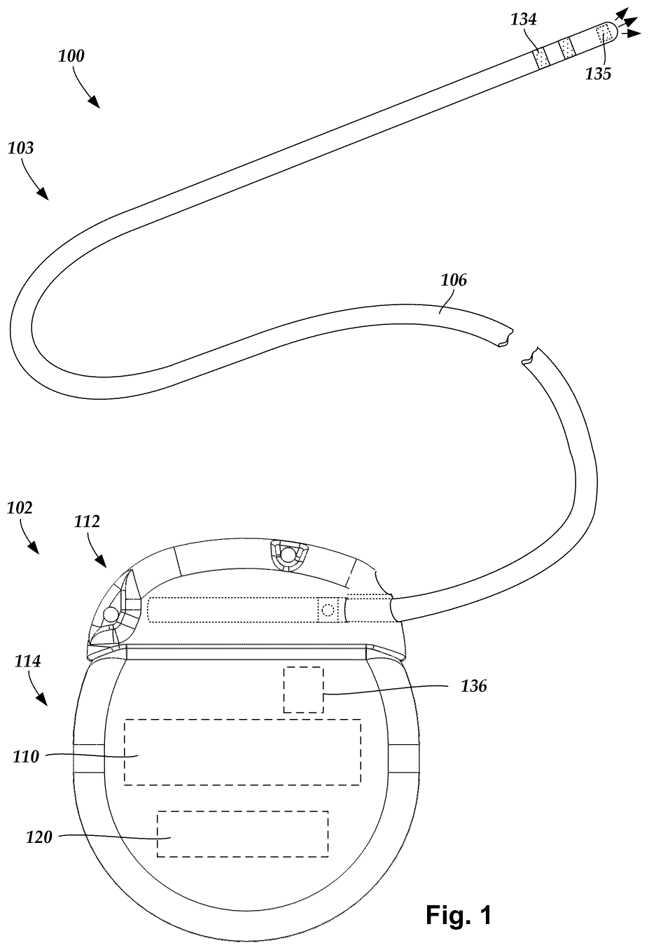

FIG. 1 illustrates schematically one embodiment of a photobiomodulation system 100. In at least some embodiments, the photobiomodulation system includes a control module (e.g., a stimulator) 102 and a lead 103 coupleable to the control module 102. The lead 103 includes one or more lead bodies 106. It will be understood that the lead 103 can include any suitable number of lead bodies including, for example, one, two, three, four, five, six, seven, eight or more lead bodies 106.

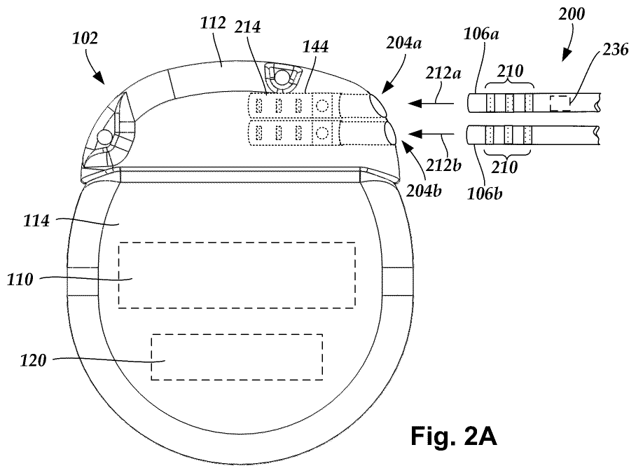

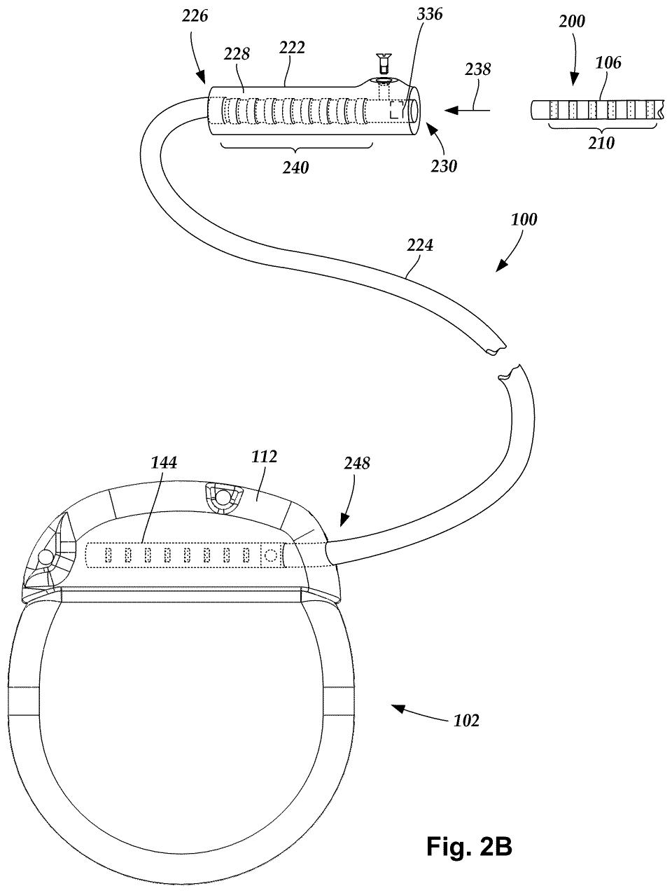

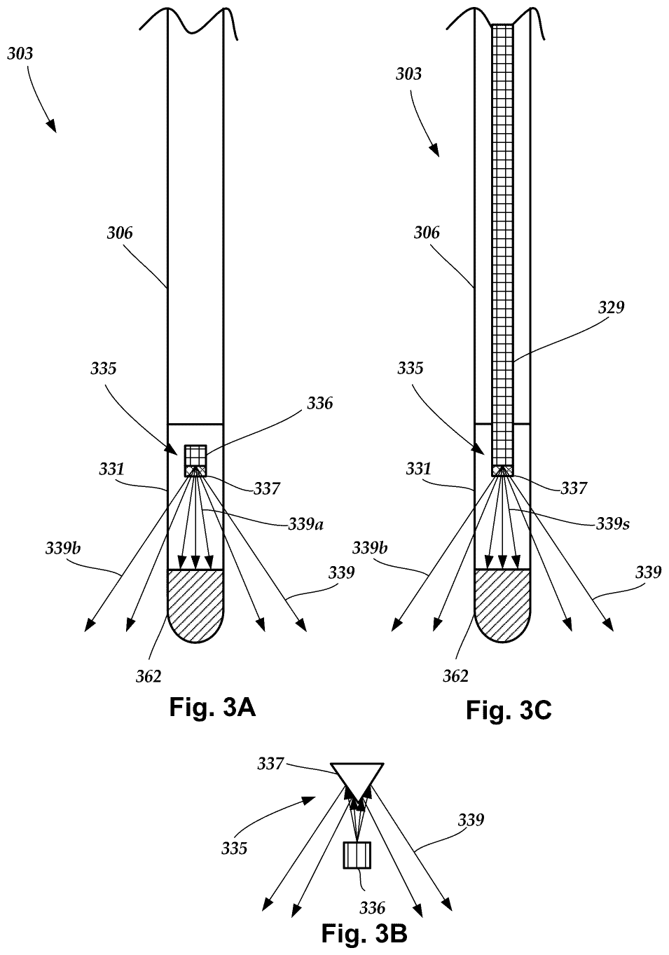

At least one light emitter 135 is provided at a distal end of the lead 103. In at least some embodiments, the light emitter 135 can include a light source. In other embodiments, the light emitter 135 can be a distal end of an optical waveguide (e.g., optical waveguide 329 of FIG. 3) and a light source can be disposed in the control module (e.g., optional light source 136 of FIG. 1), lead (e.g., optional light source 236 of FIG. 2A), or lead extension (e.g., optional light source 336 of FIG. 2B) or any other suitable components of the biomodulation system. Examples of suitable light sources include, but are not limited to, a light emitting diode (LED), laser diode, organic light emitting diode (OLED), lamp, or the like or any combination thereof. Examples of optical waveguides and light sources can be found in the references cited herein.

Optionally, the lead can also include one or more electrodes 134 disposed along the lead body 106, and one or more terminals (e.g., 210 in FIG. 2A-2B) disposed along each of the one or more lead bodies 106 and coupled to the optional electrodes 134 by conductors (not shown). In at least some embodiments, one or more terminals (e.g., 210 in FIG. 2A-2B) may also be used to convey electrical signals to a light source that acts as the light emitter 135 by conductors (not shown) extending along the lead.

The lead 103 can be coupled to the control module 102 in any suitable manner. In some embodiments, the lead is permanently attached to the control module 102. In other embodiments, the lead can be coupled to the control module 102 by a connector (e.g., connector 144 of FIG. 2A). In the embodiment illustrated in FIG. 2A, the lead 103 is shown coupling directly to the control module 102 through the connector 144. In at least some other embodiments, the lead 103 couples to the control module 102 via one or more intermediate devices, as illustrated in FIG. 2B. For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the photobiomodulation system 100 includes multiple elongated devices disposed between the lead 103 and the control module 102, the intermediate devices may be configured into any suitable arrangement.

The control module 102 can include, for example, a connector housing 112 and a sealed electronics housing 114. In at least some embodiments, an electronic subassembly 110 and an optional power source 120 are disposed in the electronics housing 114. In at least some embodiments, a control module connector 144 is disposed in the connector housing 112. In at least some embodiments, the control module connector 144 is configured and arranged to make an electrical connection between the lead 103 and the electronic subassembly 110 of the control module 102.

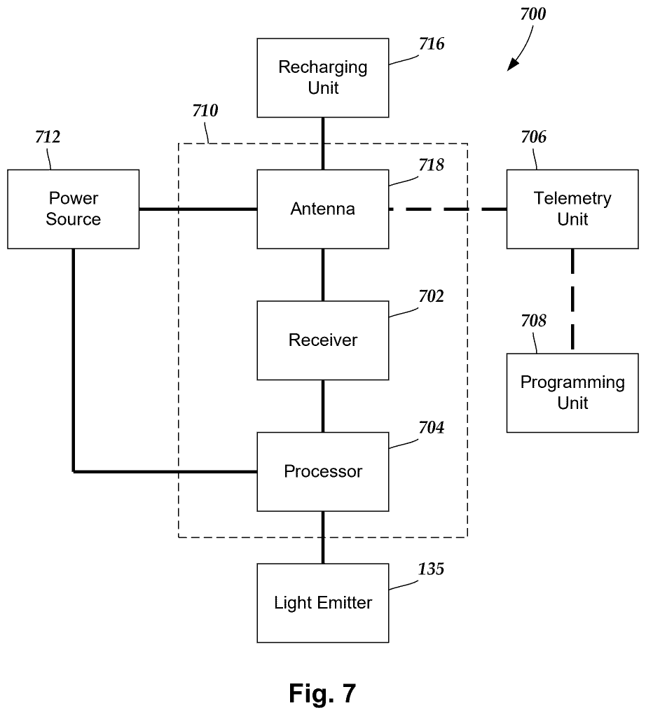

The photobiomodulation system or components of the photobiomodulation system, including the lead 103 and the control module 102, are typically implanted into the body of a patient. The photobiomodulation system can be used for a variety of applications including, but not limited to brain photobiomodulation, deep brain photobiomodulation, neural photobiomodulation, spinal cord photobiomodulation, muscle photobiomodulation, and the like. Photobiomodulation performed by the PBM system may include optical modulation, photostimulation, or any other suitable forms of optical stimulation or any combination thereof. Although not shown in FIG. 1, the photobiomodulation system 100 may also include a medical imaging system (e.g., imaging system 790 of FIG. 7).

When the lead includes the optional electrodes 134, the electrodes can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. Any suitable number of electrodes 134 can be disposed on the lead in various embodiments, including, for example, one, two, three, four, five, six, seven, eight, nine, ten, eleven, twelve, or more electrodes 134.

The one or more lead bodies 106 are made of a non-conductive, biocompatible material such as, for example, silicone, polyurethane, polyetheretherketone (“PEEK”), epoxy, and the like or combinations thereof. The one or more lead bodies 106 may be formed in the desired shape by any process including, for example, molding (including injection molding), casting, and the like.

One or more terminals (e.g., 210 in FIGS. 2A-2B) are typically disposed along the proximal end of the one or more lead bodies 106 of the photobiomodulation system 100 (as well as any splitters, lead extensions, adaptors, or the like) for electrical connection to corresponding connector contacts (e.g., 214 in FIGS. 2A-2B). In at least some embodiments, the connector contacts are disposed in connectors (e.g., 144 in FIGS. 1-2B; and 222 FIG. 2B) which, in turn, are disposed on, for example, the control module 102 (or a lead extension, a splitter, an adaptor, or the like). In at least some embodiments, electrically conductive wires, cables, or the like (not shown) extend from the terminals to the light emitter 135 or optional one or more electrodes 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end of one or more elongated devices 200 configured and arranged for coupling to one embodiment of the control module connector 144. The one or more elongated devices may include, for example, one or more of the lead bodies 106 of FIG. 1, one or more intermediate devices (e.g., a splitter, the lead extension 224 of FIG. 2B, an adaptor, or the like or combinations thereof), or a combination thereof.

In at least some embodiments, the control module connector 144 defines at least one port into which a proximal end of the elongated device 200 can be inserted, as shown by directional arrows 212a and 212b. In FIG. 2A (and in other figures), the connector housing 112 is shown having two ports 204a and 204b. The connector housing 112 can define any suitable number of ports including, for example, one, two, three, four, five, six, seven, eight, or more ports.

In at least some embodiments, the control module connector 144 also includes a plurality of connector contacts, such as connector contact 214, disposed within each port 204a and 204b. In at least some embodiments, when the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed on the paddle body 104 of the lead 103. Each of the terminals 210 can couple to the light emitter 135 or one or more of the optional electrodes 134. Examples of connectors in control modules are found in, for example, U.S. Pat. Nos. 7,244,150 and 8,224,450, which are incorporated by reference.

FIG. 2B is a schematic side view of another embodiment of the photobiomodulation system 100. In at least some embodiments, the photobiomodulation system 100 includes a lead extension 224 that is configured and arranged to couple one or more elongated devices 200 (e.g., one of the lead bodies 106 of FIG. 1, a splitter, an adaptor, another lead extension, or the like or combinations thereof) to the control module 102. In the embodiment illustrated in FIG. 2B, the lead extension 224 is shown coupled to a single port 204 defined in the control module connector 144. Additionally, in the embodiments illustrated in FIG. 2B, the lead extension 224 is shown configured and arranged to couple to a single elongated device 200. In alternate embodiments, the lead extension 224 is configured and arranged to couple to multiple ports 204 defined in the control module connector 144 (e.g., the ports 204a and 204b of FIG. 1), or to receive multiple elongated devices 200 (e.g., both of the lead bodies 106 of FIG. 1), or both.

In at least some embodiments, a lead extension connector 222 is disposed on the lead extension 224. In the embodiment illustrated in FIG. 2B, the lead extension connector 222 is shown disposed at a distal end 226 of the lead extension 224. In at least some embodiments, the lead extension connector 222 includes a connector housing 228. In at least some embodiments, the connector housing 228 defines at least one port 230 into which terminals 210 of the elongated device 200 can be inserted, as shown by directional arrow 238. Each of the terminals 210 can couple to the light emitter 135 or one or more of the optional electrodes 134. In at least some embodiments, the connector housing 228 also includes a plurality of connector contacts, such as connector contact 240. In at least some embodiments, when the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

The implantation of components of the photobiomodulation system can be performed by a surgeon or other clinician. The surgeon can make use of a medical imaging system to guide the implantation. The medical imaging system can include a fluoroscope or any other suitable imaging device. The surgeon can use medical imaging during implantation of the photobiomodulation lead to control the depth (or position) of the lead during the implantation procedure. However, it may be difficult to identify the tip of a photobiomodulation lead because often the components for photobiomodulation at the distal end of the lead may not be clearly seen in the image. It can be important to know the position of the tip of the photobiomodulation lead to avoid or reduce further tissue damage or access to tissue regions that produce side effects. Described herein are leads, systems, and methods for aiding the surgeon or other clinician in determining the depth or position of the lead (and, in particular, the distal end of the lead) during implantation.

FIG. 3A is a side view of a distal end portion of one embodiment of a photobiomodulation lead 303. The photobiomodulation lead 303 includes a lead body 306, a light emitter 335, and a radiopaque tip 362. The radiopaque tip 362 is visible on images, such as fluoroscope images, and can aid the surgeon in determining the depth (or position) of the distal tip of the photobiomodulation lead 303 during implantation. The radiopaque tip 362 can be made of any suitable biocompatible, radiopaque material(s) including, but not limited to, metals, metal alloys, barium sulfate, bismuth compounds, or the like or any combination thereof. In at least some embodiments, the radiopaque tip 362 can be made of metal or metal alloy and be electrically coupled to one of the terminals 210 (FIG. 2A) so that the radiopaque tip can also be used as a tip electrode.

In at least some embodiments, the light emitter 335 includes a light source 336 and a light distribution element 337, as illustrated in FIG. 3A. The light source 336 can be, for example, a light emitting diode (LED), organic light emitting diode (OLED), laser diode, lamp, or any other suitable light source. The light distribution element 337 can be an integral part of the light source 336, one or more components added or attached to the light source, one or more components separated from the light source, or any combination thereof.

As illustrated in FIG. 3A, the radiopaque tip 362 is distal to the light emitter 335. The radiopaque tip 362 is often opaque to light emitted by the light emitter 335 and can block, absorb, or reflect (or any combination of these effects) light directed toward it. To address this structural issue, the light emitter 335 emits light 339 in a cone (i.e., having a conical shape), as illustrated in FIG. 3A. In at least some embodiments, as illustrated in FIG. 3A, the radiopaque tip 362 is disposed in a path of a portion 339a of the emitted light 339. Another portion 339b of the light 339 passes distally beyond the radiopaque tip 362 to provide photobiomodulation to the tissue.

The light distribution element 337 includes one or more optical elements that modify the light emitted by the light source 336 into a conical shape. Examples of suitable optical elements include, but are not limited to, one or more of a cylindrical lens, a prism, or any other suitable optical element(s), or any combination thereof.

FIG. 3B illustrates another embodiment of a light distribution element 337 that takes the form of a cone mirror. The light source 336 emits light toward the cone mirror (i.e., light distribution element 337) which reflects the light into a conical shape (i.e., a cone). In at least some of the embodiments utilizing the arrangement illustrated in FIG. 3B, the radiopaque tip 362 may not be in the path of any of the emitted light 339 reflected by the cone mirror (i.e., light distribution element 337). In other embodiments utilizing the arrangement illustrated in FIG. 3B, the radiopaque tip 362 may be in the path of a portion of the emitted light 339 reflected by the cone mirror (i.e., light distribution element 337).

FIG. 3C illustrates the distal end portion of another embodiment of a photobiomodulation lead 303 that can be the same as the photobiomodulation lead 303 of FIG. 3A except that the light emitter 335 is a distal end of an optical waveguide 329, such as an optical fiber or the like. The optical waveguide 329 carries light directly or indirectly (e.g., through a chain of optical waveguides) from a light source, such as, for example, a light source in the control module (e.g., optional light source 136 of FIG. 1), lead (e.g., optional light source 236 of FIG. 2A), or lead extension (e.g., optional light source 336 of FIG. 2B) or elsewhere in the photobiomodulation system. Examples of photobiomodulation systems and optical stimulation systems, which can be modified as described herein to emit light in a conical shape, with optical waveguides and light sources can be found in the references cited herein. In at least some embodiments, the light distribution element 337 can include, for example, at least one cylindrical lens, at least one prism, or any other suitable optical components or the like or any combination thereof. In at least some embodiments, the light distribution element 337 can also include the distal tip of the optical waveguide 329 that can be arranged to emit light in a cone or emit light that, in combination with one or more optical elements, has a conical shape.

With respect to the light emitters 335 of FIGS. 3A-3C, in at least some embodiments, the light emitter 335 is disposed in an enclosure 331 that is at least partially transparent (e.g., at least 50%, 75%, 80%, 85%, 95%, or 99% transparent) to the light emitted by the light emitter 335 (or to one or more desirable wavelengths or wavelength ranges of light emitted by the light emitter). In at least some embodiments, the enclosure 331 is made of glass, quartz, epoxy, polymer, or other material that is at least partially transparent to visible or infrared light of one or more wavelengths or wavelength ranges that is emitted by the light emitter 335 for photobiomodulation of the tissue of the patient. In at least some of these embodiments, the enclosure 331, optionally, in combination with the lead body 306, radiopaque tip 362, or both, forms a hermetic, waterproof, or water resistant chamber for the light emitter 335.

In at least some embodiments, the light is emitted by the light emitter 335 with a conical shape (i.e., in a cone) having an aperture of at least 40, 30, 20, or 15 degrees. The distance between the light emitter 335 and the radiopaque tip 362 may determine or influence the amount of the light 339 that passes beyond the radiopaque tip. In at least some embodiments, the distance between the light emitter 335 and the radiopaque tip 362 is greater than the diameter of the portion of the radiopaque tip facing the light emitter.

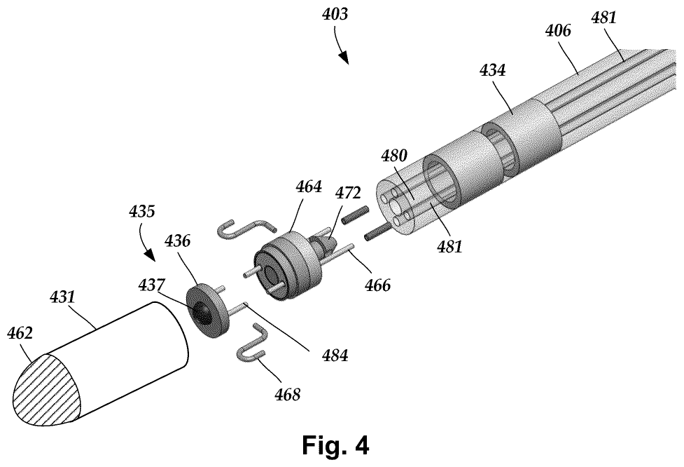

FIG. 4 is an exploded view of the distal end of one embodiment of a lead 403 with a lead body 406, a light emitter 435 having a light source 436 and a light distribution element 437, a radiopaque tip 462, and an enclosure 431. The lead 403 also includes two optional electrodes 434 disposed along the lead body 406.

FIG. 4 also illustrates one embodiment of an arrangement for electrically and mechanically coupling the light emitter 435 to the lead body 406 and remainder of the lead 403. The lead 403 includes a feedthrough assembly 464, feedthrough pins 466 that are used to electrically couple the light source to the terminals (e.g., terminals 210 of FIG. 2A) of the lead, an anchor 472 for coupling the light emitter and other elements to the lead body, contacts 484 of the light source 436, conductive cables 468 to electrically couple the contacts to the feedthrough pins, a lumen 480 for receiving the anchor 472, and one or more peripheral lumens 481 for carrying conductors (not shown), such as conductive wires or cables. Additional details of these elements can be found in U.S. Pat. No. 10,335,607, which is incorporated herein by reference in its entirety. Other photobiomodulation systems that can be modified to include the radiopaque tip and the emission of light in cone can be found in the references cited herein.

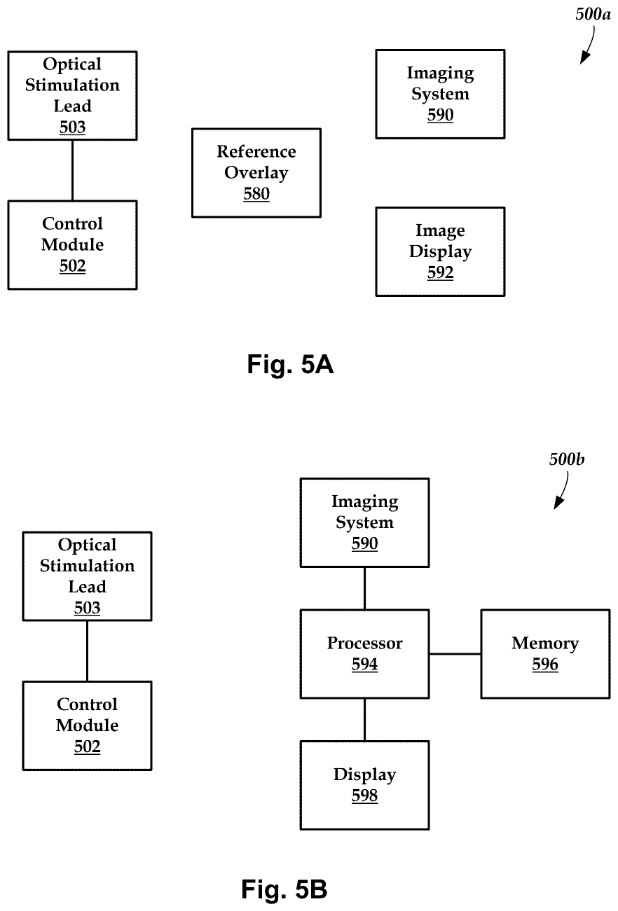

Other methods and systems for guiding implantation of a photobiomodulation lead into tissue (e.g., for depth control) utilize known dimensions of the photobiomodulation lead. FIGS. 5A and 5B are a block diagrams of two embodiments of a system 500a, 500b for guiding implantation of a photobiomodulation lead into tissue of a patient (e.g., for depth or position control).

In the embodiment of FIG. 5A, the system 500a includes a photobiomodulation lead 503, a control module 502, a reference overlay 580, an imaging system 590, and an image display 592, such as a light box or any other suitable arrangement for displaying images from the imaging system 590. The photobiomodulation lead 503 can be any suitable photobiomodulation lead, such as the photobiomodulation lead 103 of FIG. 1 or any of the photobiomodulation or optical stimulation leads described in the references cited herein. The control module 502 may be, for example, the control module 102 of FIG. 1 or any control module described in the references cited herein.

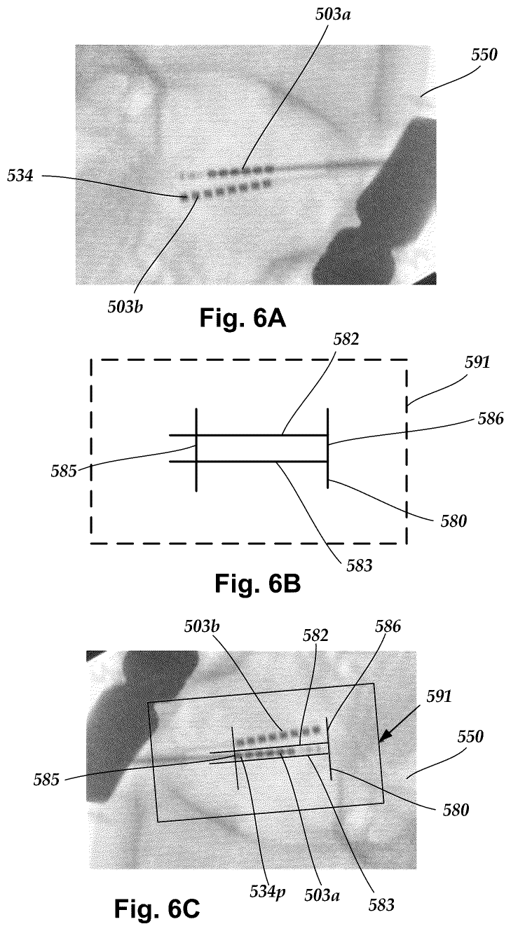

The imaging system 590 can be any suitable medical imaging system including, for example, a medical imaging system having a fluoroscope. As the photobiomodulation lead 503 is inserted into the tissue of the patient, the medical imaging system can be used to obtain an image 550 (FIG. 6A) of the distal end portion of the photobiomodulation lead 503 within the tissue of the patient. FIG. 6A illustrates one example of a fluoroscopic image 550 of a photobiomodulation lead 503a and an electrical stimulation lead 503b inserted into the tissue of a patient. The electrodes 534 on the electrical stimulation lead 503b provide a guide to the end of the electrical stimulation lead 503b within the tissue indicating, for example, the depth of the insertion. In contrast, the end of the photobiomodulation lead 503a can be difficult to identify due to the lack of components that are sufficiently radiopaque.

A reference overlay 580, which represents portions of the photobiomodulation lead 503a, is used to identify the distal tip of the photobiomodulation lead. In this embodiment, the reference overlay 580 can be a physical object 591, such as a transparency overlay (e.g., reference overlay lines, as described below, printed on a transparent plastic sheet), that can be placed or projected onto the image from the imaging system and aligned with visible features of the photobiomodulation lead 503a, as illustrated in FIG. 6C.

FIG. 6B illustrates one embodiment of the reference overlay 580. The reference overlay 580 includes a first bounding line 582, a second bounding line 583 parallel to the first bounding line, a reference line 585 intersecting the first and second bounding lines, and an end line 586 intersecting the first and second bounding lines.

The first bounding line 582, second bounding line 583, reference line 585, and end line 586 of the reference overlay 580 are arranged based on the actual dimensions and arrangement of the photobiomodulation lead 503a. The first and second bounding lines 582, 583 represents opposing sides of the photobiomodulation lead 503a. The reference line 585 represents a position of a lead landmark, such as an electrode or marking. The end line 586 represent a position of the distal end (i.e., tip) of the photobiomodulation lead 503a. Different types of photobiomodulation leads may have different reference overlays. In at least some embodiments, a scale of the reference overlay 580 can be altered with the ratio of 1) the distance between the first and second bounding lines 582, 583 and 2) the distance between the reference line 585 and the end line 586 remaining the same even when the values of these distances are changed due to the scaling.

FIG. 6C illustrates the reference overlay 580 placed on an image 550 of a photobiomodulation lead 503a. The first and second bounding lines 582, 583 are aligned with the longitudinal edges of the photobiomodulation lead 503a in the image 550. The distance between the first and second bounding lines 582, 583 represents the width of the photobiomodulation lead 503a at the scale of the image 550. If the first and second bounding lines 582, 583 of the reference overlay 580 do not align with the longitudinal edges 506a, 506b of the photobiomodulation lead 503a in the image 550, the scale of the image can be altered to fit the reference overlay or the scale of the reference overlay 580 can be altered (e.g., a new reference overlay at a different scale is obtained or the scale of a projection of the reference overlay can be altered) to fit the image.

The reference line 585 is aligned with a preselected lead landmark 587 that will be visible in the image 550. For example, as illustrated in FIG. 6C, the lead landmark 587 is the proximal edge of the proximal-most electrode 534p of the photobiomodulation lead 503a. The preselected lead landmark 587 can be any other suitable marking or component of the photobiomodulation lead 503a such as, for example, a radiopaque or other marking disposed on or in the lead body 506 of the photobiomodulation lead, any specified electrode 534 of the photobiomodulation lead, any other component of the photobiomodulation lead that is visible in the image, or the like or any combination thereof. In addition to preselecting the lead landmark 587, the edge of the lead landmark 587, for example, the proximal or distal edge, can be preselected.

When the first and second bounding lines 582, 583 are aligned with the longitudinal edges 506a, 506b of the photobiomodulation lead 503a in the image and the reference line 585 is aligned with a preselected lead landmark 587 visible in the image, the end line 586 indicates the position of the end of the photobiomodulation lead 503a, as illustrated in FIG. 6C.

In the embodiment of FIG. 5B, the system 500b includes a photobiomodulation lead 503, a control module 502, an imaging system 590, a display 598, a processor 594, and a memory 596. The photobiomodulation lead 503 can be any suitable photobiomodulation lead, such as the photobiomodulation lead 103 of FIG. 1 or any of the photobiomodulation or optical stimulation leads described in the references cited herein. The control module 502 can be, for example, the control module 102 of FIG. 1 or a control module described in the references cited herein. The imaging system 590 can be any suitable medical imaging system including, for example, a medical imaging system having a fluoroscope.

The processor 594, memory 596, and display 598 can be any suitable processor, memory, and display, respectively. In at least some embodiments, the processor 594, memory 596, and, optionally, display 598 can be parts of a computer, a tablet, a handheld device, an augmented reality system, or other suitable device or system.

The imaging system 590 generates an image 550 that includes the photobiomodulation lead 503a. The image 550 is provided directly or indirectly to the processor 594 and memory 596. The processor 594 displays the image 550 on the display 598. In at least some embodiments, instead of a physical reference overlay, the reference overlay 580 is digitally generated by the processor 594 (or drawn by a user) over the image 550 on the display 598. This digital reference overlay 580 is the same as the physical reference overlay described above except that it is not a physical article (as acknowledged by the dotted lines in FIG. 6B.

In at least some embodiments, the system 500b includes one or more controls for a user to move the reference overlay 580 and align the first and second bounding lines 582, 583 and the reference line 585 with the corresponding features in the displayed image 550 of the photobiomodulation lead 503a. The system 500b may also include one or more controls for scaling the reference overlay 580 or the image 550.

In at least some embodiments, the system 500b includes one or more controls for a user to draw or otherwise direct generation of the first and second bounding lines 582, 583 and the reference line 585. Upon drawing or generating the first and second bounding lines 582, 583 and the reference line 585, the processor can determine and display the end line 586 based on the known dimensions of the photobiomodulation lead 503a.

In at least some embodiments, the system 500b can analyze the image 550 to determine or identify the positions of one or more of the first and second bounding lines 582, 583 and the reference line 585 using, for example, image recognition software or algorithms. Upon determining or identifying the first and second bounding lines 582, 583 and the reference line 585, the processor can determine and display the end line 586 based on the known dimensions of the photobiomodulation lead 503a. In at least some embodiments, the system 500b can include controls for adjusting any of the first or second bounding lines 582, 583 or reference line 585. In at least some embodiments, the system 500b includes one or more controls for a user to draw or otherwise direct generation of the first and second bounding lines 582, 583 or the reference line 585 when the system 500b does not, cannot, or incorrectly determines the positions of one or more of the first and second bounding lines 582, 583 and the reference line 585.

FIG. 7 is a schematic overview of one embodiment of components of an photobiomodulation system 700 including an electronic subassembly 710 disposed within a control module. It will be understood that the photobiomodulation system can include more, fewer, or different components and can have a variety of different configurations including those configurations disclosed in the stimulator references cited herein.

Some of the components (for example, a power source 712, an antenna 718, a receiver 702, and a processor 704) of the photobiomodulation system can be positioned on one or more circuit boards or similar carriers within a sealed housing of an implantable pulse generator, if desired. Any suitable power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, light is emitted by the light emitter 135 of the lead body to stimulate nerve fibers, muscle fibers, or other body tissues near the photobiomodulation system. The processor 704 is generally included to control the timing and other characteristics of the photobiomodulation system. For example, the processor 704 can, if desired, control one or more of the timing, pulse frequency, strength, duration, and waveform of the photobiomodulation. In addition, the processor 704 can select one or more of the optional electrodes to provide electrical stimulation, if desired. In some embodiments, the processor 704 selects which of the optional electrode(s) are cathodes and which electrode(s) are anodes.

Any processor can be used and can be as simple as an electronic device that, for example, produces photobiomodulation at a regular interval or the processor can be capable of receiving and interpreting instructions from an external programming unit 708 that, for example, allows modification of photobiomodulation characteristics. In the illustrated embodiment, the processor 704 is coupled to a receiver 702 which, in turn, is coupled to the optional antenna 718. This allows the processor 704 to receive instructions from an external source to, for example, direct the photobiomodulation characteristics and the selection of electrodes, if desired.

In one embodiment, the antenna 718 is capable of receiving signals (e.g., RF signals) from an external telemetry unit 706 which is programmed by the programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the photobiomodulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and the receiver 702 can be used to modify or otherwise direct the operation of the photobiomodulation system. For example, the signals may be used to modify the photobiomodulation characteristics of the photobiomodulation system such as modifying one or more of photobiomodulation duration, pulse frequency, waveform, and photobiomodulation amplitude. The signals may also direct the photobiomodulation system 700 to cease operation, to start operation, to start charging the battery, or to stop charging the battery. In other embodiments, the photobiomodulation system does not include the antenna 718 or receiver 702 and the processor 704 operates as programmed.

Optionally, the photobiomodulation system 700 may include a transmitter (not shown) coupled to the processor 704 and the antenna 718 for transmitting signals back to the telemetry unit 706 or another unit capable of receiving the signals. For example, the photobiomodulation system 700 may transmit signals indicating whether the photobiomodulation system 700 is operating properly or not or indicating when the battery needs to be charged or the level of charge remaining in the battery. The processor 704 may also be capable of transmitting information about the photobiomodulation characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A photobiomodulation lead, comprising:
a lead body having a distal end portion and a proximal end portion;
an enclosure coupled to the distal end portion of the lead body;
a light emitter disposed in the enclosure and configured to emit light transmitted distally with a conical shape; and
a radiopaque tip disposed distal to the light emitter, wherein the light emitter and radiopaque tip are spaced apart so that a portion of the light emitted with the conical shape passes distally beyond the radiopaque tip.

2. The photobiomodulation lead of claim 1, wherein the light emitter comprises a light source.

3. The photobiomodulation lead of claim 1, wherein the photobiomodulation lead further comprises an optical waveguide extending along at least the distal end portion of the lead body, wherein the light emitter comprises a distal end portion of the optical waveguide.

4. The photobiomodulation lead of claim 1, wherein the light emitter comprises at least one of a prism, a plurality of mirrors, or a cylindrical lens for distributing the light in the conical shape.

5. A method of stimulating patient tissue of the patient, the method comprising:
implanting the photobiomodulation lead of claim 1 into the patient tissue; and
emitting the light from the light emitter of the photobiomodulation lead to stimulate the patient tissue.

6. A photobiomodulation system, comprising:
the photobiomodulation lead of claim 1; and
a control module coupleable to the photobiomodulation lead, the control module comprising:
a housing, and
an electronic subassembly disposed in the housing and configured to direct the emission of the light.

7. The photobiomodulation system of claim 6, wherein the photobiomodulation lead further comprises an optical waveguide extending along at least the distal end portion of the lead body, wherein the light emitter comprises a distal end portion of the optical waveguide, the photobiomodulation system comprising a light source configured for transmitting light into the optical waveguide when directed by the electronic subassembly of the control module.

8. The photobiomodulation system of claim 7, wherein the light source is disposed in the control module.

9. The photobiomodulation system of claim 7, wherein either a) the light source is disposed in the photobiomodulation lead or b) the photobiomodulation system further comprises a lead extension coupleable to the photobiomodulation lead and the control module, wherein the light source is disposed in the lead extension.

10. A method for implanting a photobiomodulation lead, the method comprising:
introducing the photobiomodulation lead into tissue of a patient;
obtaining an image of the photobiomodulation lead in the tissue;
providing a reference overlay comprising a first bounding line, a second bounding line parallel to the first bounding line, a reference line intersecting the first and second bounding lines, and an end line intersecting the first and second bounding lines; and
determining a position of a distal end of the photobiomodulation lead by overlaying the reference overlay on the image with the first and second bounding lines aligned along opposing sides of the photobiomodulation lead in the image and the reference line positioned at a lead landmark visible in the image of the photobiomodulation lead, wherein the end line indicates the position of the distal end of the photobiomodulation lead.

11. The method of claim 10, wherein the image is a fluoroscope image.

12. The method of claim 10, wherein the reference overlay is a physical transparency overlay.

13. The method of claim 10, wherein obtaining the image comprises displaying the image on a display.

14. The method of claim 13, wherein providing a reference overlay comprises generating the reference overlay on the display.

15. The method of claim 10, further comprising altering a scale of the image or the reference overlay so that the first and second bounding lines are aligned along opposing sides of the photobiomodulation lead.

16. A system for guiding implantation of a photobiomodulation lead into tissue of a patient, the system comprising:
a display;
a memory having instructions disposed thereon; and
a processor coupled to the memory and display and configured to execute the instructions to perform actions, the actions comprising:
obtaining an image of the photobiomodulation lead in the tissue,
providing a reference overlay comprising a first bounding line, a second bounding line parallel to the first bounding line, a reference line intersecting the first and second bounding lines, and an end line intersecting the first and second bounding lines, and
determining a position of a distal end of the photobiomodulation lead by overlaying the reference overlay on the image with the first and second bounding lines aligned along opposing sides of the photobiomodulation lead in the image and the reference line positioned at a lead landmark visible in the image of the photobiomodulation lead, wherein the end line indicates the position of the distal end of the photobiomodulation lead.

17. The system of claim 16, wherein the system is an augmented reality system.

18. The system of claim 16, wherein the image is fluoroscope image.

19. The system of claim 16, wherein the actions further comprise altering a scale of the image or the reference overlay so that the first and second bounding lines are aligned along opposing sides of the photobiomodulation lead.

20. The system of claim 16, wherein the actions further comprise providing controls for a user to either a) move the reference overlay relative to the image or b) draw the reference overlay on the image.
